(19) United States
(12) Patent Application Publication
(10) Pub. No.: US 2019/0350895 A1
(43) Pub. Date: Nov. 21, 2019

(54) PHOTOCHROMATIC MODULATION WITH FLUORESCEIN FOR THE TREATMENT OF PHOTO-OCULODYNIA AND BLEPHAROSPASM

(71) Applicant: Baylor College of Medicine, Houston, TX (US)

(72) Inventors: Michael Tze-Chien Yen, Houston, TX (US); Stephen C. Pflugfelder, Houston, TX (US); Francis K. Manuel, Houston, TX (US)

(21) Appl. No.: 16/335,343

(22) PCT Filed: Sep. 22, 2017
(86) PCT No.: PCT/US17/53015

§ 371 Date: Mar. 21, 2019

Related U.S. Application Data
(60) Provisional application No. 62/404,933, filed on Oct. 6, 2016.

(51) Int. Cl. A61K 31/352; A61P 27/02; A61K 38/13; A61K 31/4725; A61K 9/00

(52) U.S. Cl. CPC A61K 31/352; A61P 27/02; A61K 9/0048; A61K 31/4725; A61K 9/0051; A61K 38/13

(57) ABSTRACT

Embodiments of the disclosure concern methods and compositions that include a substrate that comprises at least one fluorophore in sufficient amounts and delivery of the substrate to an individual in need of treatment. In specific embodiments, a sufficient amount of fluorescein is provided on a contact lens, for example, and delivered to an individual suffering from blepharospasm.

This application claims priority to U.S. Provisional Patent Application Ser. No. 62/404,933, filed Oct. 6, 2016, which is incorporated by reference herein in its entirety.

### TECHNICAL FIELD

Embodiments of the disclosure concern at least the fields of ophthalmology, neurology, cell biology, molecular biology, and medicine.

### BACKGROUND

Benign essential blepharospasm is a form of focal dystonia with involuntary contractions of the eyelid protractors. The mainstay for treatment is typically targeting the involuntary protractor spasms with either botulinum-A toxin injections or surgical myectomy. Along with the involuntary contractions, however, is a constellation of sensory symptoms that include severe photophobia or photo-oculodynia. These sensory symptoms are not relieved with the treatments targeting the eyelid spasms, and often the patients are more symptomatic as their eyelid spasms are relieved since that allows greater exposure to light. At present, the only practical treatment for photophobia or photo-oculodynia is for patients to wear darkly tinted glasses, both indoors and outdoors. This treatment is only modestly effective and can be limiting to the patients' quality of life.

The present disclosure satisfies long-felt needs in the art to treat at least focal dystonias, among other medical conditions.

### BRIEF SUMMARY

Embodiments of the disclosure include methods and compositions for the treatment of a medical condition with at least one fluorophore. In particular embodiments the medical condition is a focal dystonia, such as blepharospasm, although other medical conditions may be treated, such as photo-oculodynia, photophobia, or secondary blepharospasm from ocular surface disease. Other conditions include orofacial dystonia (Meige Syndrome) and cervicofacial dystonia, for example.

In particular embodiments, a substrate comprising one or more fluorophores is provided to an individual in need of treatment with the fluorophore. The substrate may be of any kind so long as it is capable of delivering topically a therapeutically effective amount of the fluorophore. In particular embodiments, the substrate is a scleral contact lens with the fluid reservoir filled with a fluorescein solution.

In particular embodiments, the fluorophore is provided on a substrate near the eye, such as on the glass piece(s) of eye glasses to block the particular wavelengths of light that are absorbed by the fluorophore.

In one embodiment, there is a method of treating a medical condition in one or both eyes of an individual, comprising the step of providing to the eye or eyes a therapeutically effective amount (such as 0.1%-1%, including 0.1%-1% of a solution) of a fluorophore, wherein the fluorophore is provided to the eye or eyes in and/or on a substrate that is in contact with at least part of the eye or is provided on a substrate placed in front of the eye. The medical condition may be a focal dystonia, photo-oculodynia, or photophobia, for example. In specific cases, the focal dystonia is blepharospasm. In certain cases, the individual also has keratoconjunctivitis sicca. In a specific embodiment, the fluorophore is retained continuously on the surface of the eye for the time the substrate is in contact with the eye. The fluorophore may be fluorescein, rhodamine, coumarin, cyanine, hydroxycoumarin, aminocoumarin, methoxycoumarin, cascade blue, pacific blue, pacific orange, Lucifer yellow, NBD, R-phycoerythrin, TruRed, Texas Red, Cy2, Cy3, Cy5, Cy7, or derivatives thereof. In specific embodiments, the individual is given a therapeutically effective amount of one or more other compounds, such as a drug or a dry eye solution. In specific embodiments, the drug is cyclosporine, lifitigrast, or a combination thereof. The other compound may be delivered to the individual in and/or on the substrate. The other compound may be delivered to the individual in a delivery route other than in and/or on the substrate. In specific embodiments, the substrate is in contact with the surface of the cornea or is a lens inside the eye. The substrate may be a contact lens. In specific embodiments, the substrate is an intraocular lens implant. In particular aspects, the substrate is comprised of hydrogels, silicone hydrogels, polymethyl methacrylate (PMMA), or silicone acrylates. The substrate near or in front of the eye may be eyeglasses, in particular cases. In some methods, the methods further comprise the step of diagnosing the medical condition.

In one embodiment, there is a method of treating an individual for a focal dystonia or photophobia, comprising the step of providing to the individual a therapeutically effective amount of a fluorophore. The focal dystonia may be orofacial dystonia or cervicofacial dystonia, or severe photophobia from other causes. Embodiments of the disclosure also encompass treatment of migraine, uveitis, iridocyclitis, aniridia, and/or congenital glaucoma such as may or may not be associated with severe photophobia.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description. It is to be expressly understood, however, that each of the descriptive embodiments are provided for the purpose of illustration and description only and is not intended as a definition of the limits of the present invention.

### DETAILED DESCRIPTION

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

Embodiments of the disclosure concern methods and compositions for treatment of medical conditions, particularly of the eye. In particular embodiments, benign essential blepharospasm, a form of focal dystonia with involuntary contractions of the eyelid protractors, is treated with methods and compositions of the disclosure. Typically, blepharospasm is bilateral in 75% of cases, occurs in the sixth decade, is 3 times more common in females, and progressively worsens in 75% of cases. Although the signs and symptoms of blepharospasm have been recognized and reported for many years, the underlying pathophysiology remains obscure. Thus, current treatments have been directed mainly towards symptomatic relief of the motor signs of eyelid spasm. Blepharospasm patients also experience a distinct constellation of sensory symptoms. The present disclosure provides therapies for blepharospasm and other medical conditions.

I. Fluorophore Compositions

In embodiments of the disclosure, one or more fluorophores are provided in a therapeutically effective amount to an individual in need thereof. The fluorophore may be any kind of fluorophores, but in specific embodiments the fluorophore is one or more of fluorescein, rhodamine, coumarin, cyanine, hydroxycoumarin, aminocoumarin, methoxycoumarin, cascade blue, pacific blue, pacific orange, Lucifer yellow, NBD, R-phycoerythrin, TruRed, Texas Red, Cy2, Cy3, Cy5, Cy7, or derivatives thereof. In specific embodiments the fluorophore is fluorescein. In particular embodiments the fluorophore has an absorption spectrum similar to fluorescein (460-495 nm). In some embodiments, a subset of retina photoreceptors (intrinsically photosensitive retinal ganglion cells (IPRGCs)) are associated with photophobia, and these photoreceptors are sensitive to blue light with maximal excitation with 480 nm light. Thus, in specific embodiments, methods of the disclosure may utilize fluorophores that absorb in the blue spectrum from 400-500 nm, for example at 480 m.

In certain cases when more than one fluorophore is delivered to an individual, the different fluorophores may be given at the same time or at different times. In some cases a combination of different fluorophores are provided to the individual such as a first flurophore that may or may not have a different absorption spectrum from a second fluorophore. For example, a first fluorophore with an absorption spectrum at 460-495 nm and a second fluorophore with an absorption spectrum at 400-500 nm may be provided to an individual. In specific embodiments, the different fluorophores may or may not be administered to an individual at the same time.

The fluorophore may or may not be obtained commercially.

In certain cases one or more fluorophores are given to an individual in a particular dose and/or certain dosing regimens. In specific cases the individual is given 0.1% to 1% for a dose. The dose may be in the range of 0.1-1%, 0.1-0.9%, 0.1-0.8%, 0.1-0.7%, 0.1-0.6%, 0.1-0.5%, 0.1-0.4%, 0.1-0.3%, 0.1-0.2%, 0.2-1%, 0.2-0.9%, 0.2-0.8%, 0.2-0.7%, 0.2-0.6%, 0.2-0.5%, 0.2-0.4%, 0.2-0.3%, 0.3-1%, 0.3-0.9%, 03.-0.8%, 0.3-0.7%, 0.3-0.6%, 0.3-0.5%, 0.3-0.4%, 0.4-1%, 0.4-0.9%, 0.4-0.8%, 0.4-0.7%, 0.4-0.6%, 0.4-0.5%, 0.5-1%, 0.5-0.9%, 0.5-0.8%, 0.5-0.7%, 0.5-0.6%, 0.6-1%, 0.6-0.9%, 0.6-0.8%, 0.6-0.7%, 0.7-1%, 0.7-0.9%, 0.7-0.8%, 0.8-1%, 0.8-0.9%, or 0.9-1.0%. In some cases the dose is 0.1%, 0.2%, 0.3%, 0.4%, 0.5%, 0.6%, 0.7%, 0.8%, 0.9%, or 1%. In some cases the dose is between 0.01 and 10%, including between 1 and 10%, 1 and 5%, 0.1 and 5%, 0.01 and 2.5%, 2.5 and 10%, 2.5 and 5%, and so forth. When multiple deliveries are provided to the individual, they may or may not be delivered by the same delivery route. When multiple deliveries are provided to the individual, they may or may not be provided the same doses. In such cases, a subsequent dose may be more or less than an initial or previous dose.

In specific cases the fluorophore may be provided to the individual at the same time or at a different time as another, different compound, such as a drug. The drug may be for the same medical condition or a different medical condition (including one that is directly or indirectly related to the medical condition being treated with the fluorophore). The drug may be an antibiotic, cyclosporine, lifitigrast, or a combination thereof.

The fluorophore may be provided in a formulation, such as a pharmaceutical formulation.

The fluorophore may absorb light at 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 and any range there between.

II. Substrate Compositions

In particular embodiments, the substrate comprises one or more fluorophores and is used as a delivery substrate for an individual in need of treatment with the fluorophore(s). The substrate may comprise the fluorophore on it and/or in it. The substrate may need to be placed in the eye and/or on the eye. In particular cases the substrate is configured and/or used such that it avoids the lacrimal lake of the eye.

The substrate may be comprised of any suitable material and in specific embodiments is translucent, although in alternative embodiments the substrate has at least some tint, either to protect from light or to provide color to the eyeball for aesthetic purposes (for example, blue, green, or brown). The substrate may in particular cases be comprised of rigid or soft contact lenses or spectacle lenses. In specific embodiments the substrate is shaped to conform to the eye surface or interior, and in particular cases the shape is curved to match the curvature of the eye. In particular embodiments, the shape of the substrate is specifically fitted to an individual, whereas in other cases the substrate is obtained for or by the individual off-the-shelf.

In particular embodiments, the substrate is configured to be in contact with the surface of the cornea or is a lens inside the eye. In specific embodiments, the substrate is a contact lens or an intraocular lens implant. In other embodiments, eyeglasses or lens of any kind comprise one or more fluorophores. In certain cases, the fluorophore is delivered as a gel placed on the on the eye.

In specific embodiments, the substrate is disposable after use, whereas in other cases it is not disposable. Under certain conditions, the substrate may be re-used, although in particular embodiments the substrate is for single use only. The substrate may be suitable packaged to provide a single use substrate or multiple substrates are packaged for multiple uses. In particular cases the substrate is provided to the user in a form for immediate use, whereas in other cases the user must take action to utilize the substrate in a form for immediate use. For example, in certain cases the substrate is provided to the user with fluorophore already configured onto and/or in the substrate, whereas in other cases the substrate requires exposer to the fluorophore under certain conditions prior to use.

In particular embodiments, a substrate that is utilized is a contact lens already utilized by the user for correcting vision, and the user delivers a suitable amount of fluorophore to the corrective contact lens. In other cases, the lens is not also used for corrective vision but has the sole purpose of delivering the fluorophore to the individual.

### III. Methods of Making and Use of the Compositions

In certain embodiments, the disclosure encompasses methods of making a substrate that comprises one or more fluorophores. In certain embodiments, a manufacturer applies the fluorophore(s) to the substrate, whereas in other cases a medical provider and/or a user apply the fluorophore(s) to the substrate. One aspect provides for the addition of a drop to a soft contact lens or filling the reservoir of a scleral contact lens with a fluorophore-comprising solution, which in some cases is 0.1%-1.0%.

In particular embodiments, an individual with a medical condition is provided a therapeutically effective amount of a substrate that comprises one or more fluorophores. In particular embodiments the fluorophore(s) is provided to the individual at a localized area to address a medical condition in need of localized therapy, such as having a localized diseased area. In certain embodiments, the medical condition affects the eye or, in some cases, the face. In some cases, the medical condition is focal dystonia (eye dystonia (also called blepharospasm) or orofacial dystonia or cervicofacial dystonia). In cases where the eye is affected by the medical condition, the medical condition may be photophobia (discomfort or pain to the eye(s) from light exposure), including severe photophobia that accompanies moderate to severe dry eye. In certain cases the individual also has a second medical condition, including a second medical condition that affects the eye (for example, keratoconjunctivitis sicca).

The substrate comprising the one or more fluorophores may be provided to the location in need for a sufficient duration in time to produce a therapeutic effect, whether or not the therapeutic effect is confirmed by a medical practitioner. In specific embodiments, the substrate with the fluorophore(s) is used by the individual as needed, or it may be used in particular intervals until it is no longer needed. When utilized at multiple deliveries (single use or multi-uses), the interval(s) between uses may be the same or may be different. The intervals may be on the order of minutes, hours, days, weeks, months or years. In particular embodiments, therapeutic effect requires continuous and perpetual use. Without the fluorescein, the treatment effect is lost, in particular embodiments. In the case of a contact lens, the effect could last as long as the contact lens is worn, in specific embodiments.

In each use, the substrate may be utilized by the user for a sufficient duration, and that duration may be determined by a medical practitioner based on one or more factors, including severity of the condition, age or weight of the user, and so forth. In specific embodiments, the substrate is used for a duration on the order of minutes, hours, days, and so forth. In one embodiment, an estimated duration would be for the waking hours, approximately 16 hours per day.

Although the fluorophore fluorescein is utilized in some diagnostic cases, the use of fluorescein in the context of the present disclosure is not for the purpose of diagnosis. The fluorophore/substrate combination of the present disclosure is not a part of a diagnostic evaluation, in specific embodiments. Thus, in specific embodiments the use of fluorescein in methods of the disclosure is a non-diagnosis use. Where fluorescein may be used as a topical drop into the eye, the fluorescein is removed in moments upon successive, standard blinking and washing of the eyeball by existing tears. In the present disclosure, the fluorophore is retained continuously on the surface of the eye for the time that the substrate is in contact with the eye. Thus, the fluorescein is being applied differently in standard diagnosis cases. For diagnostics, one uses only a drop, and it is transient as the fluorescein is cleared from the eye by tears and down the tear drain. For the therapy, in specific embodiments the fluorescein is contained in the space behind the contact lens, so the fluorescein is retained continuously for the time the lens is in place, or in some applications, the fluorescein may be imbedded into the contact lens material itself, in an intraocular lens, and/or on spectacle lenses; treatment can require continuous use for the therapeutic effect to be maintained, in specific embodiments.

In alternative cases, fluorescein or another fluorophore may be used to diagnose a medical condition but the substrate/fluorophore composition is used for treatment of the medical condition, including in a continuous use embodiment.

### IV. Pharmaceutical Formulation

Pharmaceutical compositions of the present disclosure may comprise an effective amount of one or more compositions including a fluorophore dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a mammal like a human, as appropriate. The preparation of a pharmaceutical composition that contains at least one fluorophore composition of the present invention and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The therapeutic compositions of the present disclosure may comprise different types of carriers depending on whether it is to be administered in solid, liquid or aerosol form, and whether it need to be sterile for such routes of administration. The present invention can be administered topically or intraocularly or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present disclosure administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In embodiments where the composition is in a liquid form, a carrier can be a solvent or dispersion medium comprising but not limited to, water, ethanol, polyol (e.g., glycerol, propylene glycol, liquid polyethylene glycol), lipids (e.g., triglycerides, vegetable oils, liposomes) and combinations thereof. In many cases, it will be useful to include isotonic agents, such as, for example, sugars, sodium chloride or combinations thereof.

Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose.

The composition must be stable under the conditions of manufacture and storage, and preserved against the contaminating action of microorganisms, such as bacteria and fungi. It will be appreciated that contamination should be kept minimally at a safe level.

V. Kits of the Disclosure

Any of the fluorophore compositions described herein may be comprised in a kit. In a non-limiting example one or more fluorophores and optionally a second compound such as a drug may be comprised in a kit. The kits will comprise any of its components in one or more suitable containers.

Depending on the fluorophore, the components of the kits may be packaged either in aqueous media or in lyophilized form. Although the kit may comprise the substrate that already comprises the one or more fluorophores, in cases where the substrate lacks the fluorophore (for example, during shipping), the user will apply the one or more fluorophores to and/or into the substrate. In such cases, in addition to the naked substrate a container means of the kits will generally include at least one vial, test tube, flask, bottle, syringe or other container means, into which the fluorophore(s) may be placed, and preferably, suitably aliquoted. Where there are more than one components in the kit, the kit also will generally contain a second, third or other additional container into which the additional components may be separately placed. However, various combinations of components may be comprised in a vial. The kits of the present disclosure also will typically include a means for containing the substate and/or fluorophore(s) components in close confinement for commercial sale. Such containers may include injection or blow-molded plastic containers into which the desired components are retained. The fluorophore components of the kit may be provided as dried powder(s), in some cases. When reagents and/or components are provided as a dry powder, the powder can be reconstituted by the addition of a suitable solvent. It is envisioned that the solvent may also be provided in another container means.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Fluorophore Treatment of Blepharospasm

The present example concerns fluorophore treatment of blepharospasm. It has been reported that up to 79% of blepharospasm patients find bright light to be the most frequent exacerbating factor of blepharospasm. Many patients wear sunglasses even on cloudy days and experience extreme photophobia in normal or even low-light conditions.

The present inventors have observed a dramatic improvement in both the sensory symptoms as well as the motor signs of blepharospasm with the use of fluorescein dye placed in the fluid reservoir (a space behind the lens (between the lens and the cornea) that is filled with fluid) behind a scleral contact lens. It was considered that the unique spectral features of fluorescein dye are responsible for the noted clinical improvement.

All patients in the study have an established diagnosis of essential blepharospasm currently managed with botulinum toxin injections. Prior to treatment with the fluorescein dye, patients are evaluated for their blink rate as determined by the number of blinks per minute. Patients are also evaluated using the Jankovic rating scale which has 2 components: 1. The severity of the eyelid spasms are rated from 0-4 (0=no spasms, 4=severe incapacitating spasms) and, 2. The frequency of spasms are rated from 0-4 (0=no spasms, 4=persistent eye closure). The sum of the severity score and the frequency score is then computed. Patients are then fitted with a scleral contact lens with normal saline solution mixed with fluorescein dye placed in the fluid reservoir. The blink rate and JRS sum score are then assessed immediately after placement of the lens.

| Patient | Pre blink-rate | Post blink-rate | Pre JRS | Post JRS |
|---|---|---|---|---|
| 1 | 80 | 10 | 7 | 1 |
| 2 | 50 | 20 | 6 | 3 |
| 3 | 60 | 40 | 6 | 4 |
| 4 | 70 | 35 | 7 | 4 |
| 5 | 70 | 49 | 7 | 3 |

In particular embodiments, the fluorescein may be applied as a coating to glasses, contact lens, or to intraocular lens implants to avoid the need of placing fluorescein within the fluid reservoir behind the scleral contact lens.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of treating a medical condition in one or both eyes of an individual, comprising the step of providing to the eye or eyes a therapeutically effective amount of one or more fluorophores, wherein one or more fluorophores are provided to the eye or eyes in and/or on a substrate that is in contact with at least part of the eye or is provided on a substrate placed in front of the eye.

2. The method of claim 1, wherein the medical condition is a focal dystonia, photo-oculodynia, or photophobia.

3. The method of claim 2, wherein the focal dystonia is blepharospasm.

4. The method of claim 1, wherein the individual also has keratoconjunctivitis sicca.

5. The method of claim 1, wherein the fluorophore is retained continuously on the surface of the eye for the time the substrate is in contact with the eye.

6. The method of claim 1, wherein the fluorophore has an absorption spectrum at 400-500 nm.

7. The method of claim 1, wherein the fluorophore has an absorption spectrum at 460-495 nm.

8. The method of claim 1, wherein the fluorophore is fluorescein, rhodamine, coumarin, cyanine, hydroxycoumarin, aminocoumarin, methoxycoumarin, cascade blue, pacific blue, pacific orange, Lucifer yellow, NBD, R-phycoerythrin, TruRed, Texas Red, Cy2, Cy3, Cy5, Cy7, or derivatives thereof.

9. The method of claim 1, wherein the individual is given a therapeutically effective amount of one or more other compounds.

10. The method of claim 9, wherein the one or more other compounds is a drug.

11. The method of claim 10, wherein the drug is cyclosporine, lifitigrast, or a combination thereof.

12. The method of claim 1, wherein the other compound is delivered to the individual in and/or on the substrate.

13. The method of claim 1, wherein the other compound is delivered to the individual in a delivery route other than in and/or on the substrate.

14. The method of claim 1, wherein the substrate is in contact with the surface of the cornea or is a lens inside the eye.

15. The method of claim 1, wherein the substrate is a contact lens.

16. The method of claim 1, wherein the substrate is an intraocular lens implant.

17. The method of claim 1, wherein the substrate is comprised of hydrogels, silicone hydrogels, polymethyl methacrylate (PMMA), or silicone acrylates.

18. The method of claim 1, wherein the substrate near the eye is eyeglasses.

19. The method of claim 1, further comprising the step of diagnosing the medical condition.

20. The method of claim 1, wherein the therapeutically effective amount is 0.1%-1%.

21. The method of claim 1, wherein the individual has migraine, uveitis, iridocyclitis, aniridia, and/or congenital glaucoma.

22. The method of claim 19, wherein the diagnosing step utilizes the fluorophore.

23. A method of treating an individual for a focal dystonia or photophobia, comprising the step of providing to the individual a therapeutically effective amount of a fluorophore.

24. The method of claim 23, wherein the focal dystonia is orofacial dystonia or cervicofacial dystonia or the severe photophobia from other causes.
